(19) United States
(12) Patent Application Publication
(10) Pub. No.: US 2023/0355292 A1
(43) Pub. Date: Nov. 9, 2023

(54) RADIAL COMPATIBLE CATHETER FOR PERI-VASCULAR FLUID INJECTION

(71) Applicant: Ablative Solutions, Inc., Wakefield, MA (US)

(72) Inventors: David R. Fischell, Fair Haven, NJ (US); Darrin James Kent, Murrieta, CA (US)

(21) Appl. No.: 17/662,343

(22) Filed: May 6, 2022

(51) Int. Cl. A61B 18/06; A61M 25/00; A61M 25/01

(52) U.S. Cl. CPC A61B 18/06; A61M 25/0084; A61M 25/0108; A61B 2018/00434

(57) ABSTRACT

An intravascular catheter for peri-vascular and/or peri-urethral tissue ablation includes multiple needles advanced through supported guide tubes which expand around a central axis to engage the interior surface of the wall of the renal artery or other vessel of a human body allowing the injection an ablative fluid for ablating tissue, and/or nerve fibers in the outer layer or deep to the outer layer of the vessel, or in prostatic tissue. Applications include renal denervation for the treatment of hypertension, atrial fibrillation, congestive heart failure, tissue ablation for COPD, BPH and prostate cancer and prevention of restenosis after balloon angioplasty or stent implantation and other disorders.

### FIELD

Some aspects of the disclosure are applicable to the field of devices to advance a needle like structure for injection of fluid into a volume tissue outside of the inside wall of a target vessel of a human body. Applications include renal denervation for the treatment of hypertension, atrial fibrillation, congestive heart failure, tissue ablation for COPD, BPH and prostate cancer and prevention of restenosis after balloon angioplasty or stent implantation and other disorders.

### BACKGROUND

Since the 1930s it has been known that injury or ablation of the sympathetic nerves in or near the outer layers of the renal arteries (Renal Denervation) can dramatically reduce high blood pressure. As far back as 1952, alcohol has been used for tissue ablation based renal denervation in animal experiments. Specifically Robert M. Berne in “Hemodynamics and Sodium Excretion of Denervated Kidney in Anesthetized and Unanesthetized Dog” Am J Physiol, October 1952 171:(1) 148-158, describes painting alcohol on the outside of a dog's renal artery to produce denervation.

Because of the similarities of anatomy, for the purposes of this disclosure, the term target vessel will refer here to the renal artery, for hypertension or congestive heart failure (CHF) applications, to the urethra for BPH and prostate applications and to the bronchia of the lungs for COPD applications.

Recent technology for renal denervation include energy delivery devices using radiofrequency or ultrasound energy, such as Simplicity® RF ablation catheter from Medtronic, the ultrasound ablation based system from Recor and the Peregrine® chemical denervation catheter from Ablative Solutions.

There are a number of limitations of the Simplicity® system for RF energy delivery as is does not allow for efficient circumferential ablation of the renal sympathetic nerve fibers. If circumferential RF energy were applied in a ring segment from within the renal artery (energy applied at intimal surface to kill nerves in the outer adventitial layer) this could lead to even higher risks of renal artery stenosis from the circumferential and transmural thermal injury to the intima, media and adventitia. Finally, the “burning” of the interior wall of the renal artery using RF ablation can be extremely painful. The long duration of the RF ablation renal denervation procedure requires sedation and, at times, extremely high doses of morphine or other opiates, and anesthesia close to general anesthesia, to control the severe pain associated with repeated burning of the vessel wall. Thus, there are numerous and substantial limitations of the current approach using RF-based renal sympathetic denervation. Similar limitations apply to ultrasound or other energy delivery techniques.

The Bullfrog® micro infusion catheter described by Seward et al in U.S. Pat. Nos. 6,547,803 and 7,666,163, which uses an inflatable elastic balloon to expand a single needle against the wall of a blood vessel, could be used for the injection of a chemical ablative solution such as alcohol but it would require multiple applications as those patents do not describe or anticipate the circumferential delivery of an ablative substance around the entire circumference of the vessel. The greatest number of needles shown by Seward is two and the two needle version of the Bullfrog® would be hard to miniaturize to fit through a small guiding catheter to be used in a renal artery. If only one needle is used, controlled and accurate rotation of any device at the end of a catheter is difficult at best and could be risky if the subsequent injections are not evenly spaced. This device also does not allow for a precise, controlled and adjustable depth of delivery of a neuroablative agent. This device also may have physical constraints regarding the length of the needle that can be used, thus limiting the ability to inject agents to an adequate depth, particularly in diseased renal arteries with thickened intima. Another limitation of the Bullfrog® is that inflation of a balloon within the renal artery can induce possible late vessel stenosis due to balloon injury of the intima and media of the artery, as well as causing endothelial cell denudation.

Jacobson and Davis in U.S. Pat. No. 6,302,870 describe a catheter for medication injection into the interior wall of a blood vessel. While Jacobson includes the concept of multiple needles expanding outward, each with a hilt to limit penetration of the needle into the wall of the vessel, his design depends on rotation of the tube having the needle at its distal end to allow it to get into an outward curving shape. The hilt design shown of a small disk attached a short distance proximal to the needle distal end has a fixed diameter which will increase the total diameter of the device by at least twice the diameter of the hilt so that if the hilt is large enough in diameter to stop penetration of the needle, it will significantly add to the diameter of the device. Using a hilt that has a greater diameter than the tube, increases the device profile, and also prevents the needle from being completely retracted back inside the tubular shaft from which it emerges, keeping the needles exposed and potentially allowing accidental needlestick injuries to occur. For either the renal denervation or atrial fibrillation application, the length of the needed catheter would make control of such rotation difficult. In addition, the hilts, which limit penetration, are a fixed distance from the distal end of the needles. There is no built in adjustment on penetration depth which may be important if one wishes to selectively target a specific layer in a vessel or if one needs to penetrate all the way through to the volume past the adventitia in vessels with different wall thicknesses. Jacobson also does not envision use of the injection catheter for denervation. Finally, FIG. 3 of the Jacobson patent shows a sheath over expandable needles without a guide wire and the sheath has an open distal end which makes advancement through the vascular system more difficult. Also, because of the hilts, if the needles were withdrawn completely inside of the sheath they could get stuck inside the sheath and be difficult to push out.

As early as 1980, alcohol has been shown to be effective in providing renal denervation in animal models as published by Kline et al in “Functional re-interiorvation and development of supersensitivity to NE after renal denervation in rats”, American Physiological Society 1980:0363-6110/80/0000-0000801.25, pp. R353-R358. Kline states that “95% alcohol was applied to the vessels to destroy any remaining nerve fibers. Using this technique for renal denervation, we have found renal norepinephrine concentration to be over 50% depleted (i.e. <10 mg/g tissue) two weeks after the operation.” Again in 1983 in the article “Effect of renal denervation on arterial pressure in rats with aortic nerve transaction” Hypertension, 1983, 5:468-475, Kline again publishes that a 95% alcohol solution applied during surgery is effective in ablating the nerves surrounding the renal artery in rats. Drug delivery catheters such as that by described by Jacobson which are designed to inject fluids at multiple points into the wall of an artery have existed since the 1990s.

McGuckin in U.S. Pat. No. 7,087,040 describes a tumor tissue ablation catheter having three expandable tines for injection of fluid that exit a single needle. The tines expand outward to penetrate the tissue. The McGuckin device has an open distal end that does not provide protection from inadvertent needle sticks from the sharpened tines. In addition, the McGuckin device depends on the shaped tines to be of sufficient strength so that they can expand outward and penetrate the tissue. To achieve such strength, the tines would have to be so large in diameter that severe extravascular bleeding could occur when the tines would be retracted back following fluid injection for a renal denervation application. There also is no workable penetration limiting mechanism that will reliably set the depth of penetration of the distal opening from the tines with respect to the interior wall of the vessel, nor is there a preset adjustment for such depth. For the application of treating liver tumors, the continually adjustable depth of tine penetration may make sense since multiple injections at several depths might be needed. However, for renal denervation, the ability to accurately set the penetration depth so as to not infuse the ablative fluid too shallow and injure the media of the renal artery or too deep and thus miss the nerves that are in the adventitial and peri-adventitial layers of the renal artery.

Although alcohol has historically been shown to be effective as a therapeutic agent for renal denervation and is indicated by the FDA for use in the ablation of nerves, there is need for an intravascular injection system specifically designed for the peri-vascular circumferential ablation of sympathetic nerve fibers in the outer layers around the renal arteries with sufficient penetration depth to accommodate variability in vessel wall thicknesses and to account for the fact that many renal artery nerves are situated at some distance outside of the artery's adventitia.

In U.S. Pat. No. 9,056,185, issued Jun. 16, 2015, U.S. Pat. No. 9,179,962, issued Nov. 10, 2015, U.S. Pat. No. 9,254,360, issued Feb. 9, 2016, U.S. Pat. No. 9,301,795, issued Feb. 9, 2016, U.S. Pat. No. 9,320,850, issued Apr. 26, 2016, U.S. Pat. No. 9,526,827, issued Dec. 27, 2016, U.S. Pat. No. 9,539,047, issued Jan. 10, 2017, U.S. Pat. No. 9,554,849, issued Jun. 3, 2014, U.S. Pat. No. 9,795,441, issued Oct. 24, 2017, U.S. Pat. No. 10,118,004, issued Nov. 6, 2018, U.S. Pat. No. 10,226,278, issued Mar. 12, 2019, U.S. Pat. No. 10,350,392, issued Jul. 16, 2019, U.S. Pat. No. 10,405,912, issued Sep. 10, 2019, U.S. Pat. No. 10,485,951, issued Nov. 26, 2019, and U.S. Pat. No. 10,576,246, issued Mar. 3, 2020, which are hereby incorporated by reference in their entirety, Fischell et al show multiple embodiments of a fluid delivery catheter for injection of a fluid into the peri-vascular space of a vessel of a human body. Mechanisms shown by Fischell et al in U.S. Pat. No. 9,931,046, issued Apr. 3, 2018, U.S. Pat. No. 9,949,652, issued Apr. 3, 2018, U.S. Pat. No. 10,022,059, issued Apr. 3, 2018, U.S. Pat. No. 10,420,481, issued Sep. 24, 2019, and U.S. Pat. No. 10,517,666, issued Dec. 31, 2011, which are hereby incorporated by reference in their entirety, are used to advance electrodes with or without fluid injection capability into and beyond the inside wall of a target vessel for nerve sensing, electrical stimulation and energy based tissue ablation.

Together, these two groups of patents form the “Fischell Patents” for reference throughout this specification and are hereby incorporated by reference in their entirety. In some embodiments described therein, the Fischell Patents use needle guiding elements in the form of guide tubes to support the advancement and penetration through the inside wall of a target vessel of needles/wires with sharpened distal ends. Such a structure can be important to allow use of small diameter needles/wires that will not cause blood loss when retracted following use in a blood vessel.

Throughout this specification any of the terms ablative fluid, ablative solution and/or ablative substance will be used interchangeably to include a liquid or a gaseous substance delivered into a volume of tissue in a human body with the intention of damaging, killing or ablating nerves or tissue within that volume of tissue.

Also throughout this specification, the term inside wall or interior surface applied to a blood vessel, vessel wall, artery or arterial wall mean the same thing which is the inside surface of the vessel wall, inside of which is the vessel lumen. Also the term injection egress is defined as the distal opening in a needle from which a fluid being injected will emerge. With respect to the injection needle, either injection egress or distal opening may be used here interchangeably.

The terminology “deep to” a structure is defined as beyond or outside of the structure so that “deep to the adventitia” refers to a volume of tissue outside of the adventitia of an artery.

The term peri-vascular refers to the volume of tissue outside of the inside wall of a target vessel. For an artery this includes the media, external elastic lamina, adventitia and peri-advential tissue.

### SUMMARY

The use of guide tubes as needle guiding elements of the catheters, such as the Peri-vascular Tissue Ablation Catheter (PTAC) of U.S. Pat. No. 9,179,962, are disclosed in the “Fischell Patents” that include Fischell et all U.S. Pat. No. 9,056,185, issued Jun. 16, 2015, U.S. Pat. No. 9,179,962, issued Nov. 10, 2015, U.S. Pat. No. 9,254,360, issued Feb. 9, 2016, U.S. Pat. No. 9,301,795, issued Apr. 5, 2016, U.S. Pat. No. 9,320,850, issued Apr. 26, 2016, U.S. Pat. No. 9,526,827, issued Dec. 27, 2016, U.S. Pat. No. 9,539,047, issued Jan. 10, 2017, and U.S. Pat. No. 9,554,849, issued Jun. 3, 2014, which are hereby incorporated by reference in their entirety. Such guiding elements are essential for the support of small diameter needles to access the volume of tissue deep to the inside wall of a target vessel.

Some aspects of the disclosure include a Fluid Injection Catheter (FIC) that uses injector tubes with distal needles advanced and retracted through guide tubes. The FIC comprises a number of embodiments that improve upon the prior art.

The Peri-vascular Tissue Ablation Catheter PTAC 100 as shown in FIG. 3 of Fischell et al U.S. Pat. Nos. 9,179,962, 9,254,360, 9,301,795, 9,320,850, 9,526,827, 9,539,047, and 9,554,849 shows the distal portion assembly including a central buttress with support ramps for the guide tubes and the outer tube extension that lies outside the central buttress to connect it to the proximal end of the distal tapered section of the catheter. Some aspects of the disclosure include improvements related to extending the outer tube extension in the distal direction over a significant portion of the tapered section of the catheter.

Some aspects of the disclosure includes a two layer outer tube extension with a slit flap opening structure forming a window in the outer layer of the outer tube extension that increases the reliability for extension and retraction of the guide tubes.

Some aspects of the disclosure includes embodiments that can be of small enough diameter to be placed through a 6 French guiding catheter, i.e. 6 French compatible. This is accomplished through specific design specifications that will allow a functional set of guide tubes and injector tubes with distal needles with suitable radiopacity, but with an overall diameter of less than 0.07 inches in diameter. These include use of injector tubes with OD of less than 0.01″ and ID of less than 0.007″ with internal radiopaque wires of less than 0.0055″ diameter. Additional embodiments include modifications of the injector tubes with distal needles to allow for a smaller diameter.

Some aspects of the disclosure includes embodiments having a weld joint for the proximal ends of radiopaque wires inserted into the lumens of the injector tubes to provide longitudinal stability for the radiopaque wires with respect to the distal needles.

Some aspects of the disclosure includes a 2 layer outer tube catheter shaft to increase flexibility while maintaining pushability.

Some aspects of the disclosure include structures such as alignment holes to secure alignment between the two layers of the outer tube extension and a pin and slot mechanism to align the central buttress component with the outer tube extension though which the injector tubes with distal needles are advanced and retracted.

Some aspects of the disclosure include a Fluid Injection Catheter (FIC) with a dual layer outer tube extension where the outer layer includes a slotted flap to improve the reliability of advancing and retracting the guide tubes.

Some aspects of the disclosure include an outer tube extension with a greater than 5 mm distal extension that secures the catheter to a distal tapered section.

Some aspects of the disclosure include a pin on the central buttress to align with a slot in the inner layer of the outer tube extension to align radially and longitudinally the central buttress with the openings in the outer tube extension. The slot also allows fixing this alignment when the outer layer of the outer tube extension is shrunk down onto the inner layer.

Some aspects of the disclosure include a proximal weld joint for the proximal ends of the radiopaque wires located inside the injector tubes to prevent significant distal motion of the radiopaque wires with respect to the injector tubes.

Some aspects of the disclosure include a section of multilumen catheter attached inside the main injection lumen of the FIC to prevent proximal motion of the radiopaque wires.

Some aspects of the disclosure include proper scaling of the inner tube, middle tube, outer tube, guide tubes and injector tubes as well as the associated radiopaque elements to allow the FIC to be compatible with a 6 French guiding catheter.

Some aspects of the disclosure including removing a portion of the radially outward portion of a section of each of two or more guide tubes to reduce the overall outside diameter of the portion of the FIC where the guide tubes are separated.

In some embodiments, a catheter for fluid delivery into tissue outside of an interior wall of a target vessel of a human body is provided. The catheter can include a catheter body comprising at least two openings in a distal portion of the catheter body and a central axis extending in a longitudinal direction. In some embodiments, the catheter body comprises a fluid injection lumen. In some embodiments, each of the at least two openings in the distal portion of the catheter body comprises an opening cover including at least one slit. The catheter can include at least two needle guiding elements adapted to advance distally and expand outwardly through the opening covers of the at least two openings in the distal portion of the catheter body toward the interior wall of the target vessel. The catheter can include at least two injection needles adapted to be advanced outwardly through the at least two needle guiding elements to penetrate the interior wall of the target vessel. In some embodiments, the at least two injection needles having a distal opening for fluid delivery into the tissue outside of the interior wall of the target vessel.

In some embodiments, the opening cover comprises a hole. In some embodiments, the at least one slit comprises a proximal slit. In some embodiments, the at least one slit comprises a longitudinal slit. In some embodiments, the distal portion of the catheter body comprises two layers including an inner layer and an outer layer. In some embodiments, the opening covers are formed in the outer layer. In some embodiments, a portion of the catheter body further comprises three concentric tubular structures including an outer tube, a middle tube and an inner tube. In some embodiments, the middle tube is adapted to move longitudinally with respect to the outer tube. In some embodiments, the inner tube is adapted to move longitudinally with respect to the middle tube. In some embodiments, a proximal portion of at least one of the three concentric tubular structures is formed from a metal hypotube. In some embodiments, the catheter can include at least one radiopaque marker located on at least one of the following: the catheter body, at least one needle guiding element, or at least one injection needle. In some embodiments, the slits in the opening covers increase the reliability for extension and retraction of the at least two needle guiding elements. In some embodiments, the slits in the opening covers guide the at least two needle guiding elements through the opening covers. In some embodiments, the slits in the opening covers protect the at least two needle guiding elements from surface damage as the at least two needle guiding elements are advanced and retracted from the catheter body. In some embodiments, the catheter can include a distal tapered section, wherein the distal portion of the catheter body is coupled to the distal tapered section over a length of at least 5 mm.

In some embodiments, a catheter for fluid delivery into tissue outside of an interior wall of a target vessel of a human body is provided. The catheter can include a catheter body comprising at least two openings in the distal portion of the catheter body and a central axis extending in a longitudinal direction. In some embodiments, the catheter body comprises a fluid injection lumen. The catheter can include at least two openings in the distal portion of the catheter body, each opening comprising an opening cover comprising a hole and a proximal slit. The catheter can include at least two injection needles adapted to be advanced outwardly through the holes in the opening covers of the at least two openings to penetrate the interior wall of the target vessel. In some embodiments, the at least two injection needles have a distal opening for fluid delivery into the tissue outside of the interior wall of the target vessel.

In some embodiments, the catheter can include at least two needle guiding elements adapted to advance distally and expand outwardly through the opening covers of the at least two openings, wherein the at least two injection needles are adapted to be advanced outwardly through the at least two needle guiding elements. In some embodiments, the distal portion of the catheter body comprises two layers including an inner layer and an outer layer. In some embodiments, the opening covers are formed in the outer layer and at least two openings are formed in the inner layer.

In some embodiments, a catheter for fluid delivery through into tissue outside of an interior wall of a target vessel of a human body is provided. The catheter can include a catheter body comprising three openings in the distal portion of the catheter body and a central axis extending in a longitudinal direction. In some embodiments, the catheter body comprises a fluid injection lumen. In some embodiments, each of the three openings in the distal portion of the catheter body comprise an opening cover comprising a hole and a longitudinal slit. The catheter can include three needle guiding elements adapted to advance distally and expand outwardly through the holes in the opening cover of the three openings in the distal portion of the catheter body toward the interior wall of the target vessel. The catheter can include three injector tubes with distal injection needles adapted to be advanced outwardly through the three needle guiding elements to penetrate the interior wall of the target vessel. In some embodiments, the three injection needles have a distal opening for fluid delivery into the tissue outside of the interior wall of the target vessel.

In some embodiments, the longitudinal slit is proximal to the hole. In some embodiments, the opening cover protects the three needle guiding elements. In some embodiments, the distal portion of the catheter body is coupled to a distal tapered section over a length of at least 5 mm.

In some embodiments, a catheter for fluid delivery into tissue outside of an interior wall of a target vessel of a human body is provided. The catheter can include a catheter body comprising an outer tube extension having a proximal end, a central portion and a distal portion. In some embodiments, the distal portion of the catheter body comprises at least two openings. In some embodiments, the catheter body comprises a central axis extending in a longitudinal direction, wherein the catheter body comprises a fluid injection lumen. The catheter can include at least two needle guiding elements adapted to advance distally and expand outwardly through the at least two openings in the distal portion of the catheter body toward the interior wall of the target vessel. The catheter can include at least two injection needles adapted to be advanced outwardly through the at least two needle guiding elements to penetrate the interior wall of the target vessel. In some embodiments, the at least two injection needles have a distal opening for fluid delivery into the tissue outside of the interior wall of the target vessel. The catheter can include a distal tapered section having a proximal portion and a distal end. In some embodiments, the distal portion of the outer tube extension is fixedly attached to the outside of the proximal portion of the distal tapered section over a length of at least 5 mm.

In some embodiments, the at least two openings in the distal portion of the outer body comprise an opening cover comprising a hole and a proximal slit. In some embodiments, the outer tube extension comprises an inner layer and an outer layer. In some embodiments, a distal portion of the outer layer of the outer tube extension is fixedly attached to the outside of the proximal portion of the distal tapered section. In some embodiments, a portion of the catheter body further comprises three concentric tubular structures comprising an outer tube, a middle tube and an inner tube. In some embodiments, the middle tube is adapted to move longitudinally with respect to the outer tube. In some embodiments, the inner tube is adapted to move longitudinally with respect to the middle tube. In some embodiments, a proximal portion of at least one of the three tubes is formed from a metal hypotube. In some embodiments, distal portion of the outer tube extension is fixedly attached to the outside of the proximal portion of the distal tapered section over a length of at least 10 mm. In some embodiments, the catheter can include at least one radiopaque marker located on at least one of the following: the catheter body, at least one needle guiding element, or at least one injection needle.

In some embodiments, a catheter for fluid delivery into tissue outside of the interior wall of a target vessel of a human body is provided. The catheter can include a catheter body comprising an outer tube extension having a proximal end, a central portion and a distal portion including three openings. In some embodiments, the catheter body comprises a central axis extending in a longitudinal direction. In some embodiments, the catheter body comprises a fluid injection lumen. The catheter can include three guide tubes adapted to advance distally and expand outwardly through the three openings in the distal portion of the catheter body toward the interior wall of the target vessel. The catheter can include three injector tubes with distal injection needles adapted to be advanced outwardly through the three guide tubes to penetrate the interior wall of the target vessel. In some embodiments, the three injector tubes with distal injection needles have a distal opening for fluid delivery into the tissue outside of the interior wall of the target vessel. The catheter can include a distal tapered section having a proximal portion and a distal end, wherein the distal portion of the outer tube extension is coupled to the proximal portion of the distal tapered section over a length of at least 5 mm.

In some embodiments, each of the at least three openings in the distal portion of the catheter body comprises an opening cover. In some embodiments, the opening covers protect the three needle guiding elements. In some embodiments, the opening covers guide the three needle guiding elements. In some embodiments, the outer tube extension comprises an inner layer and an outer layer. In some embodiments, the outer layer of the outer tube extension is fixedly attached to the outside of the proximal portion of the distal tapered section. In some embodiments, the outer layer of the outer tube extension comprises opening covers over the three openings. In some embodiments, a portion of the catheter body further comprises three concentric tubular structures comprising an outer tube, a middle tube and an inner tube. In some embodiments, the outer tube is coupled to the outer tube extension. In some embodiments, the distal portion of the outer tube extension is coupled to the proximal portion of the distal tapered section over a length of at least 10 mm.

In some embodiments, a catheter for fluid delivery into tissue outside of an interior wall of a target vessel of a human body is provided. The catheter can include a catheter body comprising a central axis extending in a longitudinal direction. In some embodiments, the catheter body comprises an outer tube with a distal end and an outer tube extension coupled to the distal end of the outer tube. In some embodiments, the outer tube extension comprises at least two openings. In some embodiments, the catheter body comprises a fluid injection lumen. The catheter can include at least two needle guiding elements adapted to advance distally and expand outwardly through the at least two openings in the outer tube extension toward the interior wall of the target vessel. The catheter can include at least two injection needles adapted to be advanced outwardly through the at least two needle guiding elements to penetrate the interior wall of the target vessel. In some embodiments, the at least two injection needles have a distal opening for fluid delivery into the tissue outside of the interior wall of the target vessel. In some embodiments, the outer tube extension of the catheter body being formed in two layers comprising an outer layer and an inner layer.

In some embodiments, the catheter can include at least two opening covers comprising a hole and a proximal slit. In some embodiments, the opening covers are formed as part of the outer layer of the outer tube extension. In some embodiments, the catheter can include a tapered section comprising a distal end and a proximal section. In some embodiments, the outer tube extension further comprises a distal portion located distal to the at least two openings. In some embodiments, the distal portion of the outer tube extension is fixedly attached to the proximal section of the tapered section over a longitudinal length of at least 5 mm. In some embodiments, the distal portion of the outer tube extension is fixedly attached to the proximal section of the tapered section over a longitudinal length of at least 10 mm. In some embodiments, the length of the distal portion of the outer tube extension significantly improves the strength of attachment of the tapered section to the outer tube extension. In some embodiments, the distal portion of the outer tube extension is fixedly attached to the outside of the proximal section of the tapered section. In some embodiments, the outer layer of the outer tube extension forms a flap over the at least two openings. In some embodiments, the outer layer of the outer tube extension covers the at least two openings. In some embodiments, the outer layer of the outer tube extension comprises holes that provides support for the extension and retraction of the at least two needle guiding elements. In some embodiments, the outer layer of the outer tube extension comprises a hole and a longitudinal slit, wherein the longitudinal slit guides a needle guiding element of the at least two needle guiding elements toward the hole. In some embodiments, the outer layer of the outer tube extension protect the at least two needle guiding elements from surface damage. In some embodiments, a portion of the catheter body further comprises three concentric tubular structures including the outer tube, a middle tube, and an inner tube. In some embodiments, the middle tube is adapted to move longitudinally with respect to the outer tube. In some embodiments, the inner tube is adapted to move longitudinally with respect to the middle tube. In some embodiments, a proximal portion of at least one of the three concentric tubular structures is formed from a metal hypotube. In some embodiments, the at least two injection needles are non-coring needles. In some embodiments, the catheter can include at least one radiopaque marker located on at least one of the following: the catheter body, at least one needle guiding element, or at least one injection needles.

These and other features and advantages will become obvious to a person of ordinary skill in this art upon reading of the detailed description including the associated drawings and the claims.

### BRIEF DESCRIPTION OF THE DRAWINGS

FIG. 1 is a longitudinal cross-section of a distal portion of the PTAC in its open position as it would be configured for delivery of fluid into a volume of tissue outside of the inside wall of a target vessel.

FIG. 2 is a longitudinal cross section of a distal portion of the FIC showing the dual layer outer tube and outer tube extension.

FIG. 3 is a schematic view of a distal portion of the FIC.

FIG. 4 is a longitudinal cross sectional view of an embodiment of the distal end of the FIC.

FIG. 5 is a schematic view of the central buttress and its relationship to guide tubes and the core guide wire.

FIG. 6A is a schematic view shows the inner layer of an embodiment of the outer tube extension.

FIG. 6B is a schematic view showing the alignment of the inner layer of FIG. 6A with the pin of the central buttress.

FIG. 7 is a schematic view showing the distal end of the inner tube and the proximal ends of the injector tubes with the proximal ends of the radiopaque wires, that run inside the injector tubes, welded together.

FIG. 8 is a schematic view showing a close up from an area of FIG. 7 showing the proximal ends of the injector tubes with welded radiopaque wires

FIG. 9 is a schematic view of a portion of an embodiment of a FIC showing the distal end of the inner tube and the proximal portion of the injector tubes with welded radiopaque wires with a length of dual lumen catheter attached inside of the lumen of the inner tube to prevent distal motion of the radiopaque wires.

FIG. 10 shows a schematic view of the length of dual lumen catheter of FIG. 9.

FIG. 11 is a schematic view showing an alternate embodiment of the guide tubes of the FIC with reduced outsides to reduce the overall FIC diameter.

FIG. 12 is a longitudinal cross section showing an alternative configuration to welding the three radiopaque wires shown in FIG. 8 as a way to prevent distal movement of the wires.

FIG. 13 shows a longitudinal cross section of an embodiment of the injector tube with internal wire where the proximal end of the wire is circumferentially welded to the proximal end of the injector tube.

FIG. 14 is a radial end view of the proximal end of the injector tube of FIG. 13.

### DETAILED DESCRIPTION

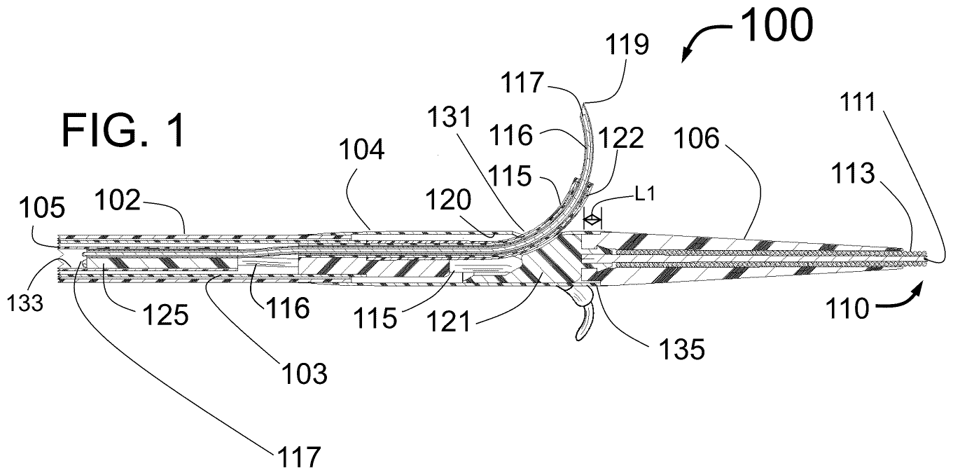

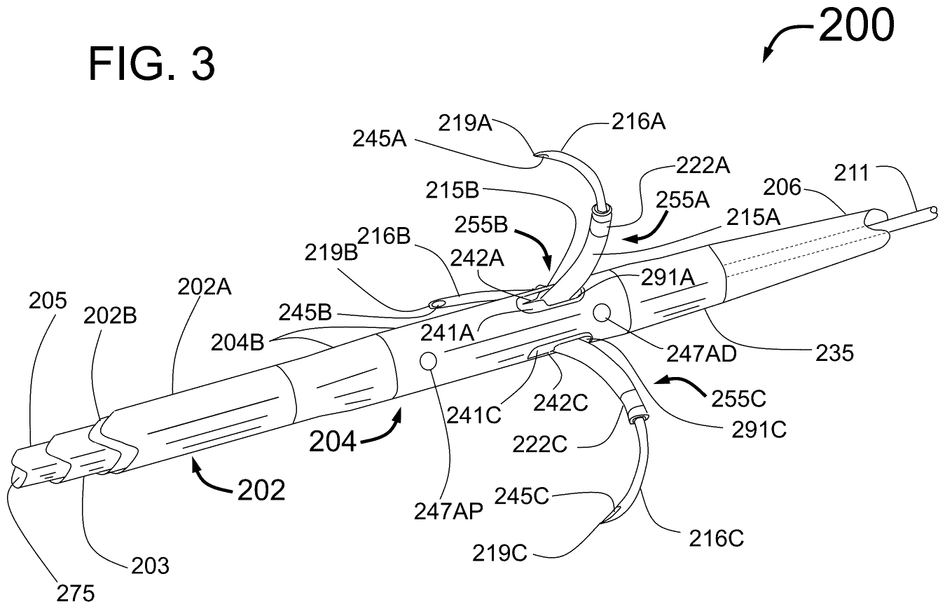

FIG. 1 is a longitudinal cross-section of a distal portion of the Peri-vascular Tissue Ablation Catheter PTAC 100. Certain embodiments and features of the PTAC are disclosed and shown in FIG. 3 of Fischell et al. U.S. Pat. Nos. 9,179,962, 9,254,360, 9,301,795, 9,320,850, 9,526,827, 9,539,047, and 9,554,849, incorporated herein by reference. The proximal end of the PTAC 100 shows the three concentric tubes, the outer tube 102, middle tube 103 and inner tube 105 which form the central portion of the PTAC 100. The outer tube 102 is attached to the outer tube extension 104 whose relatively short distal section 135 of Length L1 is attached to the tapered section 106. Based on the design, L1 would typically be less than 5 mm.

The fixed guide wire 110 with core wire 111 and outer layer 113 extends distally from the distal end of the tapered section 106. It should be noted that only part of the length of the guide wire 110 is shown in FIG. 1.

FIG. 1 shows the dual layer guide tube 115 with radiopaque marker 122 in its fully advanced position placed through the opening 131 in the outer tube extension 104. The interior surface of the outer tube extension 104 forms part of the tubular shaft 120 can be made from a stiff material such as a metal or high durometer plastic so that it will be relative rigid as the guide tubes 115 are advanced and retracted. The outer tube extension 104 is a single layer having the opening 131.

An embodiment of the PTAC 100 includes metal hypotubes connected to the proximal ends of the inner tube 105, middle tube 103 and outer tube 102.

The central buttress 121 supports the guide tube 115 as it is pushed distally. The central buttress 121 also provides radial support for the advanced guide tubes 115 that prevents the guide tubes 115 from backing away from the interior wall of the target vessel as the injector tubes 116 with sharpened needles 119 are advanced through the guide tubes 115 forward into and through the inner/interior wall of the target vessel to their desired position 2-5 mm beyond the inner wall of the target vessel. In exceptional cases, the injection needles 119 at the distal ends of the injector tubes 116 might be advanced as deep as 8 mm beyond the inner wall of the target vessel. Additional lateral support for the guide tubes 115 is provided by the sides of the openings 131 that in combination with the central buttress 121 can provide the radial and circumferential/lateral support both during guide tube 115 advancement, and as backup during delivery of the injection needles 119 through the interior wall of the target vessel. The buttress 121 may comprise a deflection surface such as a curved or linear ramp, which may in a curved embodiment correspond to the radius of curvature of the distal surface of the guide tube 115.

The inner tube 105 with fluid injection lumen 133 connects through the manifold 125 to the three injector tubes 116, thus the lumens of the injector tubes 116 are in fluid communication with the fluid injection lumen 133. The inner tube 105 and manifold 125 can slide along the longitudinal axis of the PTAC 100 inside of the middle tube 103 which is shown with uniform diameter over its length including the portion coaxially outside of the manifold 125.

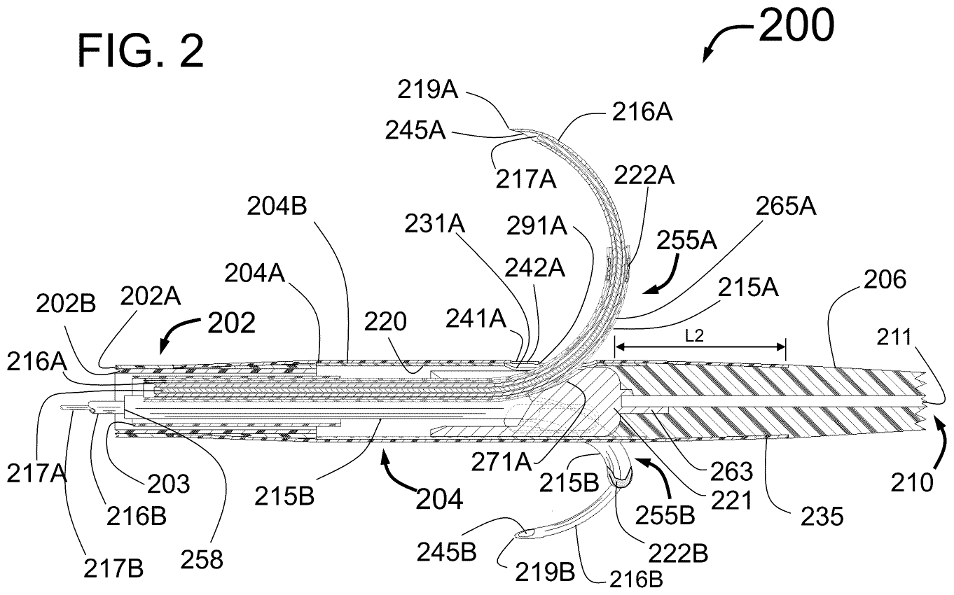

FIG. 2 is a longitudinal cross-section of the distal portion of the Fluid Injection Catheter (FIC) 200. The proximal end of FIG. 2 shows a dual layer the outer tube 202 with outer layer 202A and inner layer 202B and middle tube 203. The outer tube 202 is attached to the dual layer outer tube extension 204 with outer layer 204A and inner layer 204B which are in turn attached to the tapered section 206. The fixed guide wire 210 with core wire 211 extends distally from the distal end of the tapered section 206. It should be noted that only part of the length of the guide wire 210 is shown in FIG. 2.

FIG. 2 also shows in a longitudinal cross section, the dual layer guide tube 255A with inner layer 265A, outer layer 215A and radiopaque marker 222A in its fully advanced position placed through the opening 231A in the outer tube extension 204. The interior surface of the inner layer 204B of the outer tube extension 204 forms part of the tubular shaft 220 through which the guide tube 255A is advanced and retracted and can be made from a stiff material such as a metal or high durometer plastic so that it will be relative rigid as the guide tube 215A is advanced and retracted. The outer layer 204A includes a flap 241A with slit 242A just proximal to the hole 291A near the distal end of the outer tube extension 204. The flap 241A can cover the opening 231A in the outer tube extension 204. The flap 241A can comprises the slit 242A. The flap 241A can comprise the hole 291A. The hole 291A can be larger than the slit 242A. The hole 291A can be distal to the slit 242A. The slit 242A can be proximal to the hole 291A. The flap 241A can be a layer of material. The flap 241A can be a membrane. The flap 241A can be more flexible than another layer of the outer tube extension 204. The flap 241A can be malleable. The flap 241A can be flexible. The flap 241A can have a preformed hole 291A. The flap 241A can have the hole 291A that is formed or expanded by the penetration of the guide tube 255A. The flap 241A can have a preformed slit 242A. The flap 241A can have the slit 242A formed or expanded by the penetration of the guide tube 255A. The hole 291A can be the preferred exit for the corresponding the guide tube 255A from the outer tube extension 204. In some uses, the guide tube 255A can be misaligned and penetrate the slit 242A. The guide tube 255A can be pushed by the narrower slit 242A toward the wider hole 291A, based in part on the shape of the slit 242A and the hold 291A and/or the material of the flap 241A. The slit 242A can guide the corresponding guide tube 255A toward the hole 291A of the flap 241A.

In some embodiments, the flap 241A includes at least one slit. The flap 241A can include a proximal slit. The flap 241A can include a distal slit. The flap 241A can include a longitudinal slit. The flap 241A can include a circumferential slit. The flap 241A can include a single slit. The flap 241A can include a slit with a hole. The flap 241A can include a slit without a hole. The flap 241A can include a slit in a longitudinal direction. The flap 241A can include in a radial direction. The flap 241A can include a slit in any direction. The flap 241A can include a curved slit. The flap 241A can include an X-shaped slit. The flap 241A can include a +-shaped slit. The flap 241A can include can include intersecting slits. The flap 241A can include spaced apart slits. In some embodiments, the flap 241A include a hole 291A. The at least one slit can extend from the hole 291A. The at least one slit can intersect the hole 291A. The at least one slit and the hole 291A can be continuous. The at least one slit and the hole 291A can be discontinuous. The at least one slit and the hole 291A can be separated by a portion of the flap 241A. While a hole with a proximal slit is shown here, it is also envisioned that the opening cover could include any of the following: a single slit with or without out a hole in the longitudinal, radial or any direction, a curved slit, or an X or + shaped slit. The guide tube 255A extends through the flap 241A. The guide tube 255A can extend through both the hole 291A and the slit 242A.

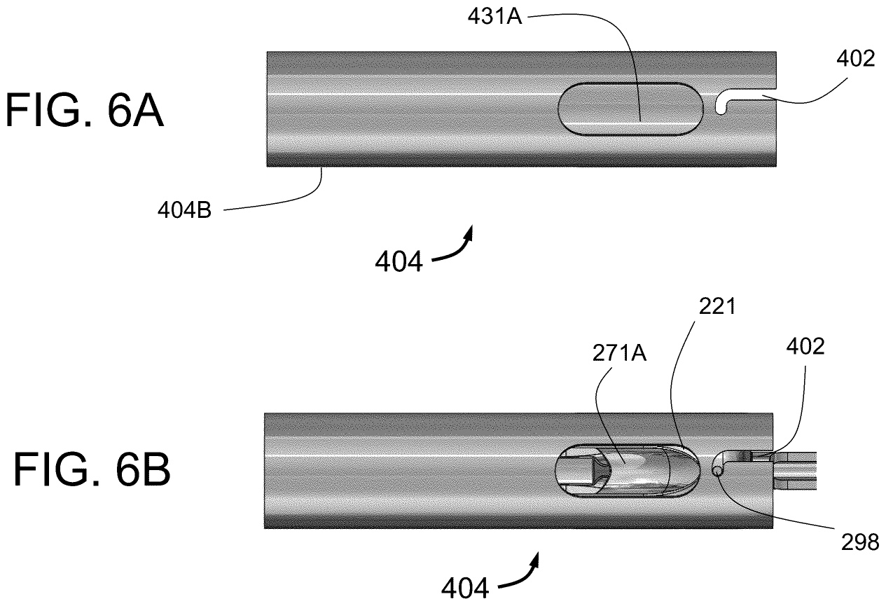

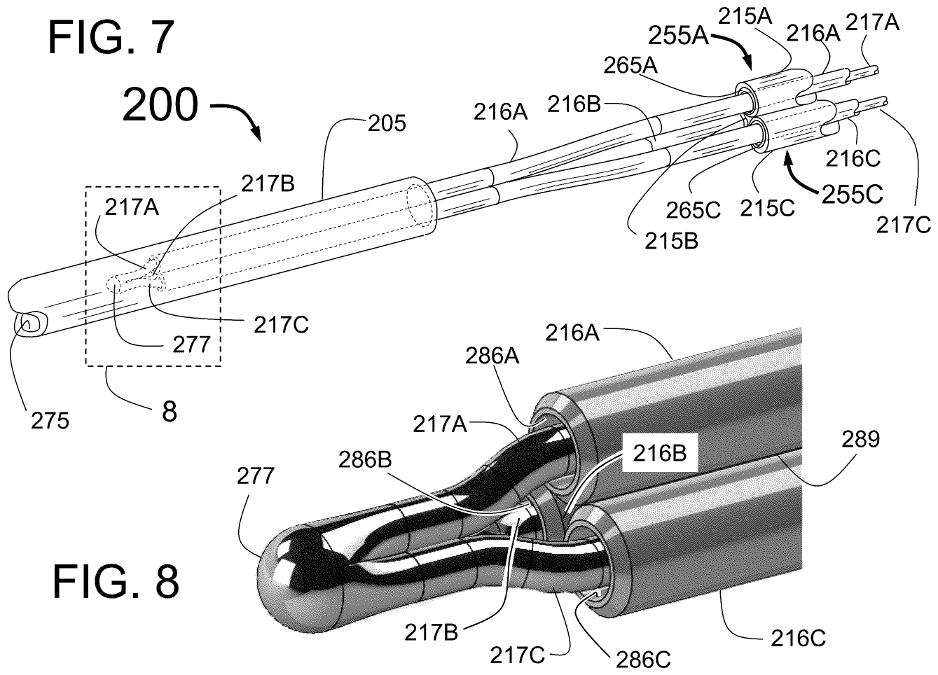

The middle tube 203 is attached to the outer layers 215A, 215B and 215C (not shown) of the guide tubes 255A, 255B and 255C (not shown). The third guide tube 255C is shown in FIG. 3. In some embodiments, the middle tube 203 attaches at its proximal end to the distal end of a metal hypotube. Together the metal hypotube connected to the middle tube 203 are used to simultaneously advance and retract the guide tubes 255A, 255B and 255C. The injector tubes 216A, 216B and 216C have sharpened non-coring needles 219A, 219B and 219C respectively at their distal ends. The injector tubes 216A, 216B and 216C (216C is shown in FIG. 3), may be advanced and retracted coaxially through the guide tubes 255A, 255B and 255C that act as needle guiding elements. The radiopaque wires 217A, 217B and 217C lie within the lumens of the injector tubes 216A, 216B and 216C respectively. Wires 216B and 216C are not shown in FIG. 2 but are shown in FIGS. 6 and 7.

In some embodiments, FIC 200 has the distal end of a metal hypotube connected to the proximal end of the outer tube 202. Together the metal hypotube and outer tube 202 form the outside of the majority of the proximal length of the FIC 200.

As shown in FIG. 2, the ramp 271A of the central buttress 221 supports the guide tube 255A as it is pushed distally and outward against the inside wall of a target vessel. The central buttress 221 also provides radial support for the fully advanced guide tube 255A that prevents the guide tube 255A from backing away from the interior wall of the target vessel as the injector tube 216A with sharpened needle 219A is advanced through the guide tube 255A into and through the inner/interior wall of a target vessel to the desired position 2-12 mm beyond the inner wall of the target vessel.

The central buttress 221 can have a distal extension 263 that is welded to the proximal end of the core wire 211 to secure it to the proximal portion of the FIC 200.

Additional lateral support for the guide tube 255A is provided by the sides of the openings 231A that in combination with the central buttress 221 can provide the radial and circumferential/lateral support both during guide tube 255A advancement, and as backup during delivery of the sharpened injection needle 219A through the interior wall of a target vessel. The ramp 271A of the buttress 221 may be formed as a curved ramp, a linear ramp or a combination of curved and linear ramp, which may in a curved embodiment correspond or be similar to the radius of curvature of the distal surface of the guide tube 225A.

While this description has been focused on guide tube 255A and injector tube 216A, this description is applicable to two or more guide tube/injector tube combinations used in the FIC 200 shown in FIGS. 2,3 and 5.

It should be noted that the embodiment of the FIC 200 of FIG. 2 has a number of important improvements over the embodiment of PTAC 100 of FIG. 1. These include:
1. The needles 219A and 219B with egress 245A and 245B respectively are non-coring needles. In some embodiments, the needle can prevent clogging of the needle by tissue.
2. The distal section 235 of the outer layer 204B of the outer tube extension has a length L2 that is significantly longer than the relatively short distal section 135 of the outer tube extension 104 of FIG. 1. This will significantly improve the strength of attachment of the tapered distal section 206 to the distal end of the FIC 200 as compared to the tapered distal section 106 to the distal end of the PTAC 100 of FIG. 1. The outer layer 204B can attach to the outside surface of the tapered distal section 206. The outer layer 204B can be attached to the outside of the tapered section 206 over a length of at least 2 mm. In some embodiments it can extend for 20 mm or more. The outer layer 204B and the tapered section 206 can form a more robust bond. The outer layer 204B and the tapered section 206 can be more securely coupled. The outer layer 204B and the tapered section 206 can comprise different materials. In some embodiments, the outer layer 204B can be more flexible than the tapered section 206. The longer attachment length can strengthen the connection between dissimilar materials of the outer layer 204B and the tapered section 206.
3. The flap 241A with slit 242A in the outer layer 204B of the outer tube extension 204 is not present in the PTAC 100 of FIG. 1. The slit flap 241A increases the reliability of advancing and retracting the guide tube 255A through the opening 231A in the outer tube extension 204. The flaps 241A, 241B and 241C can include a hole and a proximal slit. The flaps 241A, 241B and 241C can protect the guide tubes 255A, 255B and 255C that have a relatively soft outer layer 215A, 215B and 215C (not shown) from damage as the guide tubes are advanced and retracted through the openings 231A, 231B and 231C respectively. The flaps 241A, 241B and 241C can also protect the FIC 200 from intrusion into the catheter body of tissue. The guide tubes 255A, 255B and 255C can be guided toward the holes in the flaps 241A, 241B and 241C to provide a reliable means to ensure accurate deployment of the guide tubes 255A, 255B and 255C against the vessel wall. The flaps 241A, 241B and 241C can provide lateral support to the guide tubes 255A, 255B and 255C. The flaps 241A, 241B and 241C can provide circumferential alignment of the guide tubes 255A, 255B and 255C during deployment.
4. In an embodiment, the inner layer 204 B is comprised of a laser cut polyimide with three race track shaped openings 231A, 231B and 231C cut in the same circular plane, spaced 120° apart. The outer layer 204A can be formed from softer Pebax with the flaps 241A, 241B and 241C formed from the section of the outer layer 204A that lies over the openings 231A, 231B and 231C. This allows the guide tubes 255A, 255B and 255C slide against the softer Pebax of the outer layer 204A, rather than the rigid edge of the polyimide inner layer 204B; allowing the guide tubes 255A, 255B and 255C to easily be collapsed/retracted back through the openings 231A, 231B and 231C respectively.
5. The outer tube extension 204 can comprises two layers, an inner layer and an outer layer. The flaps 241A, 241B and 241C can be formed in the outer layer. The outer layer can attached to the tapered distal section for at least 5 mm. The distal portion of the outer layer of the outer tube extension can be fixedly attached to the outside of the proximal portion of the distal tapered section. The proximal catheter body can include three concentric tubular structures, an outer tube, a middle tube and an inner tube. The outer tube extension can be attached to the distal end of the outer tube. The concentric tubular structures can allow for movement for deployment of the guide tubes 255A, 255B and 255C and the injector tubes 216A, 216B and 216C. In some embodiments, the column strength of the outer body extension 204 of the FIC 200 is a critical attribute of the catheter design, wherein the outer body extension 204 must be stiff enough so that when the guide tubes 255 A, 255B and 255C (not shown) are deployed, the outer body extension 204 does not stretch or compress which could change the deployed and/or retracted position of the guide tubes 255A, 255B and 255C and/or the injector tubes 216A, 216B and 216C (not shown). In some embodiments, a material for the outer body extension with a lower coefficient of friction is also desirable.
6. In some embodiments, the outer tube 202 is formed in two layers, an outer layer 202A and an inner layer 202B as it must be flexible so that it can navigate into the renal arteries without kinking or otherwise compromising the functionality of the FIC 200.
7. In some embodiments, the outer tube 202 is constructed as a co-extrusion with the inner layer 202B being a relatively stiffer plastic of durometer 60 or higher. In some embodiments, a preferred material is 72D Pebax, which provides more column stiffness as well as has a slightly lower coefficient of friction for interfacing with the outside surfaces of the guide tubes 255A, 255B and 255C as they slide within the lumen inner layer 202B.
8. The outer layer 202A of the outer body extension 204 can be a softer and more compliant material of durometer 40 or higher. In some embodiments, a preferred material is 55D Pebax, which allows for better flexibility (without kinking) for navigating into the renal arteries.

FIG. 3 is a schematic view of a distal portion of the FIC 200 with three concentric tubes comprising the inner tube 205, middle tube 203 and dual layer outer tube 202 with inner layer 202B and outer layer 202A. The outer tube 202A is attached at its distal end to the outer layer 204A of the outer tube extension 204 with distal portion 235.

The distal portion 235 of the outer tube extension 204 outer layer 204A is attached over the proximal section of the tapered distal section 206 with core wire 211. The outer layer 204A of the outer tube extension 204 has opening covers in the form of the flaps 241A, 241C and 241B with proximal slits 242A, 242C and 242B and holes 291A, 291C and 291B that are located over the openings 231A, 231B and 231C of FIG. 2.

The three guide tubes 255A, 255B and 255C with outer layers 215A, 215B and 215C and radiopaque markers 222A, 222B and 222C are shown in their fully deployed position where they have been advanced through the holes 291A, 291B. In some embodiments, the radiopaque band 222B lies in between the outer layer 215B and inner layer 215A of the guide tube 255B. It is also envisioned that the radiopaque markers 222A, 222B and 222C could be attached outside of the outer layers 215A, 215B and 215C.

The slits 242A, 242B and 242C in the flaps 241A, 241B and 241C protect the plastic guide tube outer layers 215A, 215B and 215C from surface damage as the guide tubes 255A, 255B and 255C are advanced and retracted from within the body of the FIC 200. The flaps 241A, 241B and 241C protect the plastic guide tube outer layers 215A, 215B and 215C from damage as the catheter is advance through the vascular. The flaps 241A, 241B and 241C prevent entry of material into the catheter body through the opening 231A in the outer tube extension. The flaps 241A, 241B and 241C form a membrane over the opening 231A, 231B and 231C through which the guide tubes 255A, 255B and 255C extend. The distal portion of the middle tube 203 is attached to the proximal portion of the guide tube outer layers 215A, 215B and 215C to allow the middle tube 203 when advanced and retracted longitudinally to simultaneously advance and retract the guide tubes 255A, 255B and 255C.

The injector tubes 216A, 216B and 216C with distal non-coring needles 219A, 219B and 219C lie coaxially within and are designed to extend outward from the distal ends of the guide tubes 255A, 255B and 255C. The lumen 275 of the inner tube 205 is attached and sealed to the outsides of the injector tubes 216A, 216B and 216C. The lumen 275 is in fluid communication with the lumens of the injector tubes 216A, 216B and 216C with distal openings 245A, 245B and 245C. In some embodiments, the inner tube 205 is attached at its proximal end to a metal hypotube.

Also shown in FIG. 3 are two of the six layer lock holes 247AP and 247AD in the inner layer 204B of the outer tube extension 204 that allows the outer layer 204A of the outer tube extension 204 when heated and reflowed over the inner layer 204B to have material melt into the holes 247AP and 247AD. This locks the two layers together to prevent any motion of the inner layer 204B with respect to the outer layer 204A. The other four holes, 247BP, 247BD, 247CP and 247CD can have a similar configuration.

It is also envisioned that control handles may be used to move the guide tubes 255A, 255B and 255C with respect to the outer tube extension 204 as well as move the injector tubes 216A, 216B and 216C with sharpened needles 219A, 219B and 219C with respect to the guide tubes 255A, 255B and 255C.

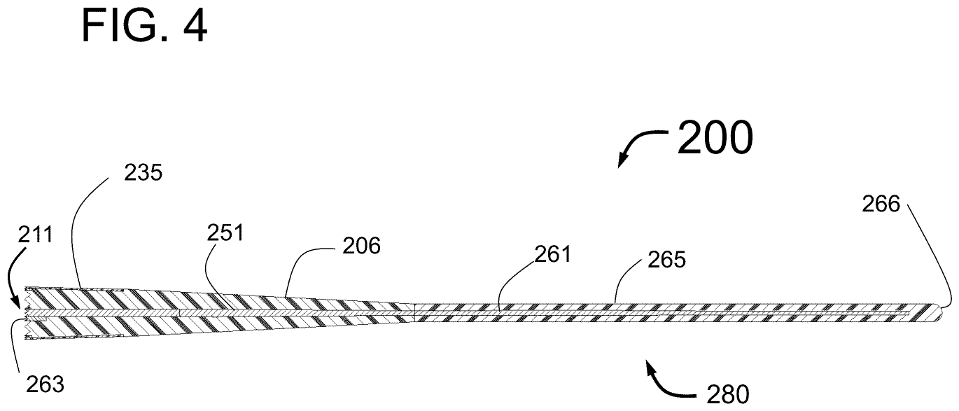

FIG. 4 is a longitudinal cross sectional view of an embodiment of the distal end of the FIC 200 with tapered section 206 and fixed guide wire 280. The fixed guide wire 280 with distal end 266 includes an outer layer 265 and a core wire 211 with tapered central portion 251 and distal portion 261. The proximal portion of the core wire 211 is attached, for example by adhesive, brazing or welding, to the central buttress distal extension 263. The distal portion 235 of the outer tube extension outer layer 204A of FIGS. 2 and 3 is attached to the outside of the tapered section 206 over a length of at least 5 mm and preferably a length of 1 cm or more that combined with the core wire 211 welded to the central buttress distal extension 263 will provide dual mechanisms to strongly secure the tapered section 206 and guide wire 280 to the central buttress 221 of FIG. 2 and hence, the proximal portion of the FIC 200. The distal portion 235 of the outer tube extension outer layer 204A of FIGS. 2 and 3 is attached to the outside of the tapered section 206 over a length of 5 mm, 6 mm, 7 mm, 8 mm, 9 mm, 10 mm/1 cm, 11 mm, 12 mm, 13 mm, 14 mm, 15 mm, 16 mm, 17 mm, 18 mm, 19 mm, 20 mm, between 5 mm and 10 mm, or any range of two of the foregoing values.

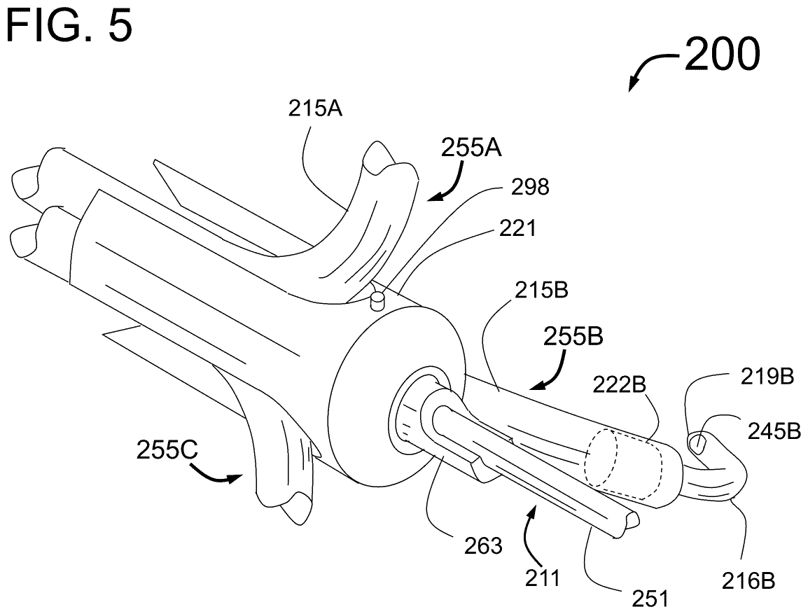

FIG. 5 is a schematic view of the central buttress 221 of the FIC 200 and its relationship to guide tubes 255A, 255B and 255C with outer layers 215A, 215B and 215C and the proximal tapered section 251 of the core wire 211. Also shown in FIG. 5 is the radiopaque band 222B of guide wire 255B and the injector tube 216B with distal non-coring needle 219B and opening 245B. The distal extension 263 of the central buttress 221 is shaped to allow insertion and subsequent attachment of the core wire 211 to the distal extension 263, by for example, adhesive, brazing, soldering or welding. The pin 298 of the central buttress 221 fits in a slot in the embodiment of the inner layer 404B of the outer tube extension 404 shown in FIGS. 6A and 6B.

FIG. 6A is a schematic view shows the inner layer 404B of an embodiment of the outer tube extension 404 with one of the three openings 431A shown of an embodiment. The slot 402 allows the pin 298 of the central buttress 221 of FIG. 5 to produce proper alignment between the central buttress 221 and the inner layer 404B in assembly. This alignment is shown in the schematic view in FIG. 6B where the pin 298 of the central buttress 221 is fully engaged into the slot 402 The slot 402 also allows a place to help maintain alignment of the outer layer 404A of the outer tube extension 404 when the outer layer 404A heat flowed and shrunk over the inner layer 404B as plastic will melt into the slot 402 and prevent movement of one layer with respect to the other. This could eliminate the need of the holes 247P and 247D shown in FIG. 3. Also shown is the ramp 271A of the central buttress 221 that will provide support for the guide tube 255A of FIGS. 2, 3, and 5 as it is advanced outward.

FIG. 7 is a schematic view showing the proximal end of the inner distal portion of the FIC 200 with injector tubes 216A, 216B and 216C with radiopaque wires 217A, 217B and 217C of the FIC 200 in relationship to the inner tube 205 and guide tubes 255A, 255B, 255C with outer layers 251A, 215B and 215C and inner layers 265A, 265B and 265C. Guide tube 255B is mostly hidden in this view. The proximal ends of the radiopaque wires 217A, 217B and 217C are welded together with the weld joint 277.

The distal portion of the lumen 275 of the inner tube 205 is sealed to the outsides of the injector tubes 216A, 216B and 216C so that fluid injected into the proximal end of the inner tube 205 will flow into the lumens 286A, 286B and 286C of FIG. 8 of the injector tubes 216A, 216B and 216C.

FIG. 8 is a schematic view showing a close up from the area 8 of FIG. 7 inside the inner tube 205 showing the proximal ends of the injector tubes 216A, 216B and 216C with radiopaque wires 217A, 217B and 217C inserted into the lumens 286A, 286B and 286C and extending in the proximal direction from the proximal ends of the injector tubes 216A, 216B and 216C. Also shown is the weld joint 277 that can prevent significant distal motion of the radiopaque wires 217A, 217B and 217C with respect to the injector tubes 216A, 216B and 216C. The radiopaque wires can be formed from a radiopaque metal or alloy such as tantalum, platinum or gold. Also shown in FIG. 8 is the space 289 between the touch point of the injector tubes 216A and 216C and the inner tube 205 of FIG. 7 with similar spaces between each pair of injector tubes 216A/216B and 216B/216C. In some embodiments, these spaces can be used for fluid flow where there is an opening in the outside of the injector tubes 216A, 216B and 216C distal to the proximal end of the injector tubes to increase the flow into the injector tubes 216A, 216B and 216C.

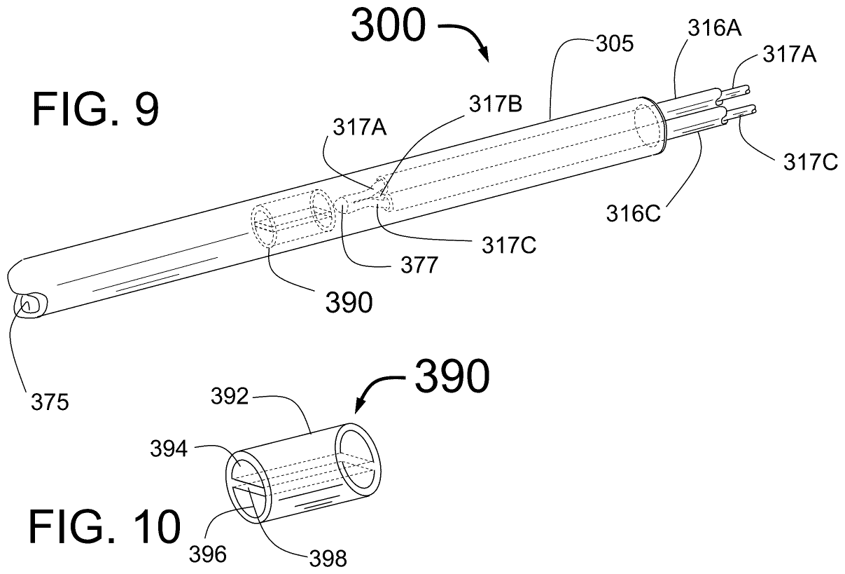

FIG. 9 is a schematic view of a portion of an embodiment of the FIC 300 showing the proximal portion of the injector tubes 316A, 316B and 316C with welded wires 317A, 317B and 317C and weld 377. In this embodiment a length of dual lumen tubing 390 is inserted into the lumen 375 of the inner tube 305. The length 390 is attached to the inside of the lumen 375 to prevent proximal motion of the welded wires 317A, 317B and 317C that could cause them to come out of the lumens of the injector tubes 316A, 316B and 316C. While FIG. 9 shows a relatively short segment of dual lumen tubing 390, it is envisioned that the length of dual lumen tubing 390 may be as short as 0.5 cm and as long as 20 cm. The length of dual lumen tubing 390 can be 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, 10 cm, 10.5 cm, 11 cm, 11.5 cm, 12 cm, 12.5 cm, 13 cm, 13.5 cm, 14 cm, 14.5 cm, 15 cm, 15.5 cm, 16 cm, 16.5 cm, 17 cm, 17.5 cm, 18 cm, 18.5 cm, 19 cm, 19.5 cm, 20 cm, between 1 cm and 5 cm, or any range of two of the foregoing values. It is also envisioned that a body of any appropriate shape, attached inside the lumen 275, just proximal to the weld 377 the that allows fluid flow around the body could provide a similar mechanism to prevent proximal movement of the wires 317A, 317B and 317C with respect to the injector tubes 316A, 316B and 316C.

FIG. 10 shows a close up schematic view of the length 390 of dual lumen catheter of FIG. 9 with tube 392 upper lumen 394 and lower lumen 396. The divider 398 between the upper lumen 394 and lower lumen 396 can engage the weld 377 if the wires 317A, 317B and 317C were to move in the proximal direction out of the lumens of the injector tubes 316A, 316B and 316C.

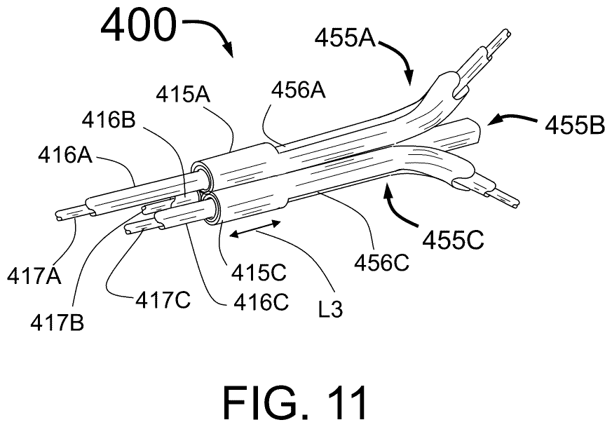

FIG. 11 is a schematic view showing an alternate embodiment of the guide tubes 455A, 455B and 455C of the FIC 400. This embodiment can have a smaller potential maximum diameter than the FIC 200 that can allow it to fit through a 6 French guiding catheter without reducing the primary diameters of the guide tubes and injector tubes.

The proximal section of length L3 of the guide tubes 455A, 455B and 455C of this embodiment of the FIC 400 are heat sealed to each other reducing the diameter compared to the more distal portion of the guide tubes 455A, 455B and 455C where they separate. It is where the guide tubes 455A, 455B and 455C separate that the diameter increases and impacts the minimum achievable diameter for the FIG. 400. To reduce this diameter, the embodiment shown in FIG. 11 has material removed from the radially outward most portion of the guide tubes labeled as 456A, 456B and 456C. The guide tube 456B is hidden in FIG. 11. This removal of material may be for a portion of the distal guide tubes. In some embodiments, this removal of material can be done for all of the guide tube length distal to the length L3. This will produce a significant reduction in the overall diameter of the FIC. 400 that could with minimal change to the overall design, allow the FIC 400 to be compatible with a 6 French guiding catheter.

In other embodiments the areas with material removed could:
1. extend all the way to the proximal end of the guide tubes 455A, 455B and 455C;
2. extend all the way to the distal end of the guide tubes 455A, 455B and 455C;
3. extend from the proximal end to the distal end of the guide tubes 455A, 455B and 455C.

In some embodiments, it is also envisioned that the guide tubes 455A, 455B and 455C can have so much material removed that they would no longer be actual tubes but more of a U shaped channel to perform as a needle guiding element. Even then the U shaped guiding elements would still provide centering of the FIC 200 inside a target vessel, still guide the injector tubes outward and still support the needles 219 A, 219B and 219C of FIG. 2 as they penetrate the inner wall of the target vessel.

In some embodiments of the U shaped channel, only a short distal portion of the guide tubes 455A, 455B and 455C would have a circular cross section. In this embodiment, the short portion could include a radiopaque marker band as shown in elements 222A and 222B of FIG. 2.

In some embodiments, one or more of the plastic layers of the guide tubes 455A, 455B and 455C could be made radiopaque using a process such as tungsten filling or by embedding a radiopaque marker between plastic layers.

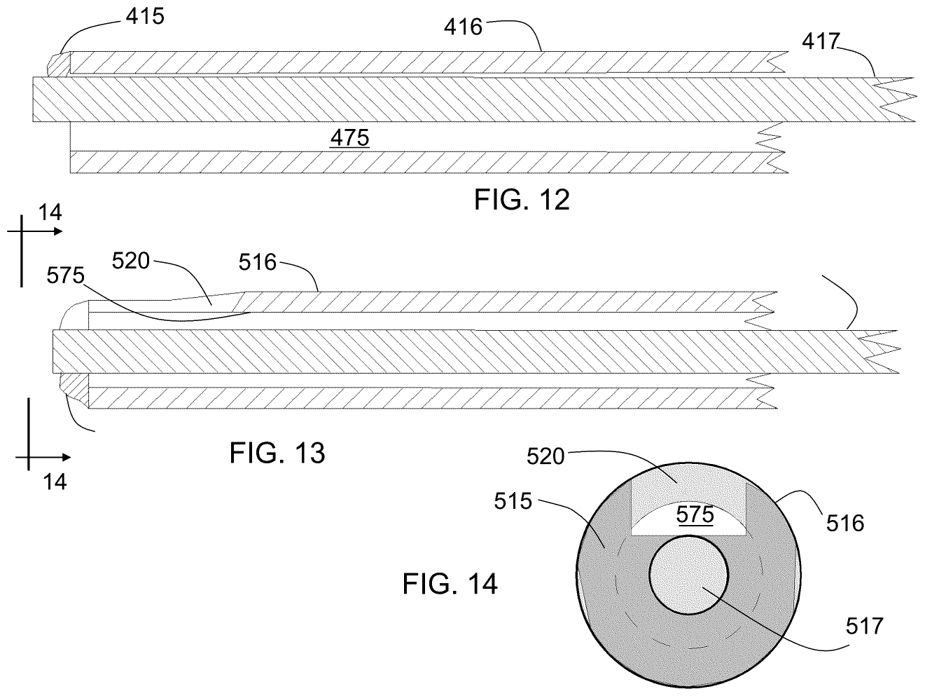

FIG. 12 is a longitudinal cross section showing an alternative configuration to welding the 3 wires 217A, 217B and 217C together, as shown in FIG. 8. Shown here is the proximal end of the injector tube 416 with internal wire 417. In this embodiment the wire 417 is spot welded with the weld 415 to the proximal end of the injector tube 416. This embodiment has the advantage of preventing both proximal and distal motion of the wire 417 with respect to the injector tube 416. This embodiment allows fluid when injected, to flow into the lumen 475 of the injector tube 416.

FIG. 13 shows a longitudinal cross section of an embodiment of the injector tube 516 with internal wire 517. In this embodiment, the proximal end of the wire 517 is circumferentially welded to the proximal end of the injector tube 516 with the weld 515. As this would by itself prevent fluid flow into the lumen of the injector tube 516, a notch or slot 520 is cut into the outside of the proximal end of the injector tube 516 with circumferentially welded wire 517. After welding or with a weld that does not cover the entire circumference of the injector tube 517, the slot 520 is formed that allows fluid to flow into the lumen 575 of the injector tube 516.

FIG. 14 is a radial end view at 14-14 of the proximal end of the injector tube 516 of FIG. 13. This shows the injector tube 516 with cut out surface 520, lumen 575, wire 517 and weld 515 that attaches the proximal end of the injector tube 516 to the proximal portion of the wire 517.

Although FIGS. 13 and 14 show a cut out at the proximal end of the injector tube 516, in some embodiments, a cut out is distal to the proximal end of the injector tube 516 with fluid flow occurring in the space between the three injector tubes e.g., the space 289 between the injector tubes 216A and 216C and the inner tube 205 of FIG. 8.

In some embodiments, a preferred material for the injector tubes 416 and 417 is a memory metal such as NITINOL. The wires 417 and 517 may be formed from the same material as the injector tubes 416 and 516 or may be formed from a radiopaque material that may include materials such as Gold, Platinum and Tantalum. The wires 417 and 517 could also be plated or coated with a radiopaque material.

While the embodiments shown in FIG. 2 through 11 show the use of three guide tubes and injector tubes, the embodiments may be configured with as few as one guide tube and injector tube and as many as six guide tubes and injector tubes. In some embodiments, two or three guide tubes and injector tubes can be provided for use in blood vessels of a human body.

Various other modifications, adaptations, and alternative designs are, of course, possible in light of the above teachings. Therefore, it should be understood at this time that within the scope of the appended claims may be practiced otherwise than as specifically described herein.

It is contemplated that various combinations or subcombinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present embodiments herein disclosed should not be limited by the particular disclosed embodiments described above. Moreover, while the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A catheter for fluid delivery into tissue outside of an interior wall of a target vessel of a human body, the catheter comprising:
a catheter body comprising at least two openings in a distal portion of the catheter body and a central axis extending in a longitudinal direction, the catheter body comprising a fluid injection lumen;
each of the at least two openings in the distal portion of the catheter body comprising an opening cover including at least one slit;
at least two needle guiding elements adapted to advance distally and expand outwardly through the opening covers of the at least two openings in the distal portion of the catheter body toward the interior wall of the target vessel; and
at least two injection needles adapted to be advanced outwardly through the at least two needle guiding elements to penetrate the interior wall of the target vessel, the at least two injection needles having a distal opening for fluid delivery into the tissue outside of the interior wall of the target vessel.

2. The catheter of claim 1, wherein the opening cover comprises a hole.

3. The catheter of claim 1, wherein the at least one slit comprises a proximal slit.

4. The catheter of claim 1, wherein the at least one slit comprises a longitudinal slit.

5. The catheter of claim 1, wherein the distal portion of the catheter body comprises two layers including an inner layer and an outer layer.

6. The catheter of claim 5, wherein the opening covers are formed in the outer layer.

7. The catheter of claim 1, wherein a portion of the catheter body further comprises three concentric tubular structures including an outer tube, a middle tube and an inner tube.

8. The catheter of claim 7, wherein the middle tube is adapted to move longitudinally with respect to the outer tube.

9. The catheter of claim 7, wherein the inner tube is adapted to move longitudinally with respect to the middle tube.

10. The catheter of claim 7, wherein a proximal portion of at least one of the three concentric tubular structures is formed from a metal hypotube.

11. The catheter of claim 1, further comprising at least one radiopaque marker located on at least one of the following:
a. the catheter body,
b. at least one needle guiding element, or
c. at least one injection needle.

12. The catheter of claim 1, wherein the slits in the opening covers increase the reliability for extension and retraction of the at least two needle guiding elements.

13. The catheter of claim 1, wherein the slits in the opening covers guide the at least two needle guiding elements through the opening covers.

14. The catheter of claim 1, wherein the slits in the opening covers protect the at least two needle guiding elements from surface damage as the at least two needle guiding elements are advanced and retracted from the catheter body.

15. The catheter of claim 1, further comprising a distal tapered section, wherein the distal portion of the catheter body is coupled to the distal tapered section over a length of at least 5 mm.

16. A catheter for fluid delivery into tissue outside of an interior wall of a target vessel of a human body, the catheter comprising:
a catheter body comprising at least two openings in the distal portion of the catheter body and a central axis extending in a longitudinal direction, the catheter body comprising a fluid injection lumen;
at least two openings in the distal portion of the catheter body, each opening comprising an opening cover comprising a hole and a proximal slit; and
at least two injection needles adapted to be advanced outwardly through the holes in the opening covers of the at least two openings to penetrate the interior wall of the target vessel, the at least two injection needles having a distal opening for fluid delivery into the tissue outside of the interior wall of the target vessel.

17. The catheter of claim 16, further comprising at least two needle guiding elements adapted to advance distally and expand outwardly through the opening covers of the at least two openings, wherein the at least two injection needles are adapted to be advanced outwardly through the at least two needle guiding elements.

18. The catheter of claim 16, wherein the distal portion of the catheter body comprises two layers including an inner layer and an outer layer.

19. The catheter of claim 18, wherein the opening covers are formed in the outer layer and at least two openings are formed in the inner layer.

20. A catheter for fluid delivery through into tissue outside of an interior wall of a target vessel of a human body, the catheter comprising:
a catheter body comprising three openings in the distal portion of the catheter body and a central axis extending in a longitudinal direction, the catheter body comprising a fluid injection lumen, wherein each of the three openings in the distal portion of the catheter body comprise an opening cover comprising a hole and a longitudinal slit;
three needle guiding elements adapted to advance distally and expand outwardly through the holes in the opening cover of the three openings in the distal portion of the catheter body toward the interior wall of the target vessel,
three injector tubes with distal injection needles adapted to be advanced outwardly through the three needle guiding elements to penetrate the interior wall of the target vessel, the three injection needles having a distal opening for fluid delivery into the tissue outside of the interior wall of the target vessel.

21. The catheter of claim 20, wherein the longitudinal slit is proximal to the hole.

22. The catheter of claim 20, wherein the opening cover protects the three needle guiding elements.

23. The catheter of claim 20, wherein the distal portion of the catheter body is coupled to a distal tapered section over a length of at least 5 mm.

24.-63. (canceled)
